(19) United States
(12) Patent Application Publication
(10) Pub. No.: US 2009/0018384 A1
(43) Pub. Date: Jan. 15, 2009

(54) Portable, Modular Transcranial Magnetic Stimulation Device

(75) Inventors: Edward S. Boyden, Cambridge, MA (US); Margaret Kim, Troy, MI (US); Gilberto Abram, Somerville, MA (US); Michael Henninger, Cambridge, MA (US)

Correspondence Address: NORMA E HENDERSON;HENDERSON PATENT LAW, 13 JEFFERSON DR, LONDONDERRY, NH, 03053, US

(73) Assignee: Massachusetts Institute of Technology, Cambridge, MA (US)

(21) Appl. No.: 12/117,896

(22) Filed: May 9, 2008

Related U.S. Application Data
(60) Provisional application No. 60/916,989, filed on May 9, 2007.

(51) Int. Cl. A61N 2/02

(52) U.S. Cl. 600/13

(57) ABSTRACT

A portable, modular transcranial magnetic stimulation device comprises a power supply unit, an energy storage unit, a charge recovery unit, a control unit, and at least one stimulation coil unit. The power supply unit generates specified voltages from a low voltage supply. The energy storage unit stores voltage received from the power supply unit until it is needed to generate a stimulation pulse and preferably includes discharge circuitry for discharging the stored voltage. The stimulation coil unit stores energy received from the energy storage unit as a strong current for application of magnetic stimulation to a patient. The charge recovery unit converts energy stored as current in the stimulation coil unit back to energy stored as voltage in the energy storage unit. The control unit controls the operation of the power supply unit, the energy storage unit, and the charge recovery unit. Biphasic, multiphasic, and monophasic stimulators may be implemented.

### RELATED APPLICATIONS

This application claims the benefit of U.S. Provisional Application Ser. No. 60/916,989, filed May 9, 2007, the entire disclosure of which is herein incorporated by reference.

### FIELD OF THE TECHNOLOGY

The present invention relates to devices for brain stimulation and, in particular, to a portable device for transcranial magnetic stimulation.

### BACKGROUND

Transcranial magnetic stimulation (TMS) is a noninvasive method for brain stimulation, first used on humans in the 1980's [A. T. Barker, R. Jalinous, and I. L. Freeston, “Non-invasive magnetic stimulation of human motor cortex”, Lancet 1 (8437), 1106 (1985)], in which a fast-changing external magnetic field induces eddy currents in the conductive tissue of the human brain, thus enabling noninvasive manipulation of neural activity over both short and long timescales. It has potential therapeutic value for treating depression, epilepsy, tinnitus, and other neurological and psychiatric disorders [M. Kobayashi and A. Pascual-Leone, “Transcranial magnetic stimulation in neurology”, Lancet neurology 2 (3), 145 (2003); A. Pascual-Leone, B. Rubio, F. Pallardo et al., “Rapid-rate transcranial magnetic stimulation of left dorsolateral prefrontal cortex in drug-resistant depression”, Lancet 348 (9022), 233 (1996); W. H. Theodore and R. S. Fisher, “Brain stimulation for epilepsy”, Lancet neurology 3 (2), 111 (2004)], but devices remain expensive and large. Existing TMS devices are powered by AC current from electrical plugs and tend to be extremely large due to the large size of the power electronics. One commercially available example is the TMS machine produced by Neuralieve (Sunnyvale, Calif.), which is wall-powered, not battery powered, and thus not wearable, portable, or modular. It is also very limited in the functionality it provides.

### SUMMARY

The present invention is a portable, modular transcranial magnetic stimulation (TMS) device. The modularity makes it possible to change the properties of the machine by exchanging parts. In a preferred embodiment, the device comprises several modular components, including a power supply unit, an energy storage unit, a charge recovery unit, a control unit, and a coil unit. The device is powered by a battery or an AC power source. Multiple devices according to the present invention can also be advantageously combined to create a larger, but still modular, TMS device.

In one aspect, the power supply unit generates specified voltages from a low voltage supply. In a preferred embodiment, it includes a flyback transformer. The energy storage unit stores voltage received from the power supply unit until it is needed to generate a stimulation pulse and preferably includes discharge circuitry for discharging the stored voltage. The energy storage unit may include a single capacitor or multiple capacitors connected in series and/or parallel. The magnetic stimulation coil unit stores energy received from the energy storage unit as a strong current for application of magnetic stimulation to a patient. The control unit controls the operation of the power supply unit, the energy storage unit, and the charge recovery unit, and is preferably implemented by means of a microcontroller. The charge recovery unit converts energy stored as current in the stimulation coil unit back to energy stored as voltage in the energy storage unit, and can be configured to determine the type and pattern of stimulation delivered to the patient. Biphasic, multiphasic, and various types of monophasic stimulators have been implemented.

### BRIEF DESCRIPTION OF THE DRAWINGS

Other aspects, advantages and novel features of the invention will become more apparent from the following detailed description of the invention when considered in conjunction with the accompanying drawing wherein:

FIG. 1 is a block diagram of an embodiment of a modular transcranial magnetic stimulation device according to the present invention;

FIG. 2 is a schematic of an exemplary embodiment of a power supply unit using a flyback transformer, according to one aspect of the present invention;

FIG. 3 is a circuit diagram of an exemplary embodiment of an energy storage circuit, according to one aspect of the present invention;

FIGS. 4A and 4B are circuit diagrams of exemplary embodiments of a multiphasic stimulator and a biphasic stimulator, respectively, according to one aspect of the present invention;

FIG. 5 depicts an exemplary timing diagram for a biphasic stimulator, according to one aspect of the present invention;

FIG. 6 is a circuit diagram of an exemplary embodiment of a monophasic stimulator, according to one aspect of the present invention;

FIG. 7 depicts a typical timing diagram for the stimulator of FIG. 6;

FIG. 8 is a circuit diagram of an exemplary embodiment of a monophasic stimulator design using the energy storage unit as part of the secondary capacitor, according to one aspect of the present invention; and

FIG. 9 depicts a typical timing diagram for the stimulator of FIG. 8.

### DETAILED DESCRIPTION

The present invention is a portable, modular transcranial magnetic stimulation (TMS) machine. It provides the ability to treat a variety of brain disorders in a systematic fashion, using a portable or wearable device. The present invention is miniaturized with respect to existing devices and is modular, so that it is easy to break down into different parts, making it easier to adapt the system on the fly for different uses. The TMS device of the present invention is portable, weighing only a few pounds. It is also inexpensive—the prototype implementation cost only a few hundred dollars to build, and a production-line version may be produced for far less. There is a high degree of modularity, which makes it easy to change properties of the TMS machine by exchanging parts. Furthermore, it permits assembling sets of stimulators, which can focus in on specific regions, via combination of multiple modular stimulators.

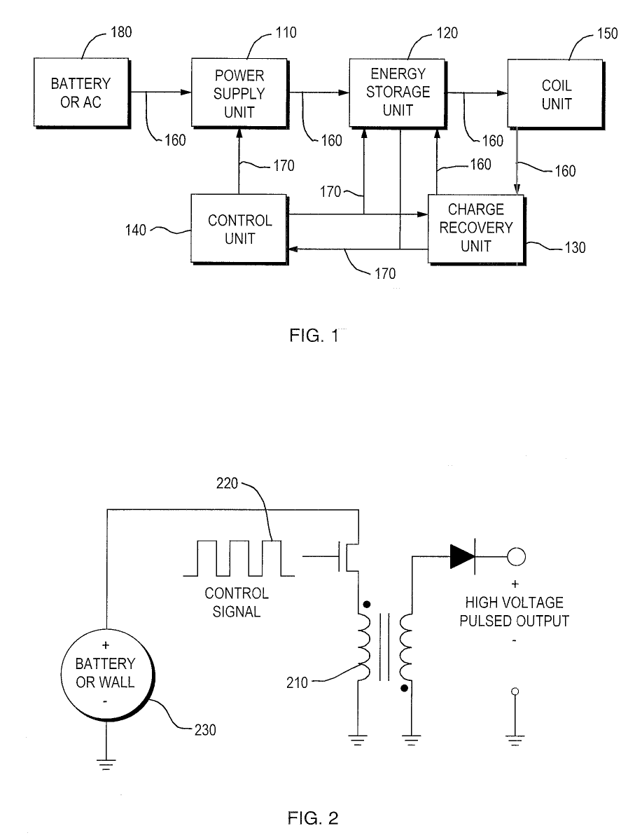

FIG. 1 is a block diagram of an embodiment of a TMS device according to the present invention. As shown in FIG. 1, the device comprises several modular components, including power supply unit 110, energy storage unit 120, charge recovery unit 130, control unit 140, and coil unit 150. In FIG. 1, power connections 160 are represented by wide arrows, control and sensing connections 170 are represented by thin arrows. The device is powered by battery or AC power source 180. Any of the modules can be changed rapidly, without the need to change any of the other parts, permitting a high degree of flexibility. A preferred embodiment comprises a battery, a flyback transformer that permits a DC voltage (e.g., battery) to charge a capacitor, a set of thyristors that optimally allow charging and discharging of the capacitor, a safety thyristor/power resistor pair that permits safe discharging, and a microcontroller to gate all the thyristors, monitor the operation of the circuit, respond to users, and store relevant protocol, patient, and other data.

The power supply unit comprises a controllable device able to generate high voltages in the range of at least 1-3 kV starting from a lower voltage power source, such as, but not limited to, a wall socket, solar panels, or batteries. High voltage generation can be achieved by use of, for example, but not limited to, transformers, boost converters, flyback transformers, or push-pull converters. The device should be able to be controlled externally, so that it can be turned on and shut off by the control unit. The output of the power supply unit is rectified to be stored in the energy storage unit.

FIG. 2 is a schematic of an exemplary embodiment of a power supply unit using a flyback transformer, according to one aspect of the present invention. This implementation employs flyback transformer 210 (e.g., the 28K074 transformer, which can create up to 4000V at 15 mA) to step up the voltage of a fast oscillating (e.g 15 to 50 kHz), low voltage (e.g. 5 to 25V), moderately high current (100s of mA) waveform 220 and is turned on and off by a microcontroller (PIC).

The source of power for the low voltage waveform is power source 230, such as, but not limited to, a wall socket adapter or battery. For example, one suitable battery configuration is a set of super-high capacity lithium ion batteries that contain storage electrodes comprising sub-100 nanometer active material particles, and can produce 2.3 Amp-hours at 3.3V, in a 70-gram package (e.g., the ANR26650M1 from A123 Systems in the present implementation). These specific batteries can store >20.000 J, enough so that five such batteries can generate O(100) full TMS pulses (100 microseconds, 1000V, 10000 A) even if all the energy is dissipated as heat (and even more if some of the energy is recovered). By powering the device with a number of such powerful, lightweight batteries, there is no need to connect the device to the wall electricity, producing a truly portable or even wearable device. These batteries can fully recharge in as little as 5 minutes, making it simple to operate with rapid cycle times, e.g. on a number of patients, or if the protocol must be repeated several times. More batteries can always be added as well; at 14 to the kilogram, they contribute to the modularity of the system. While specific battery types and configurations are described, however, it will be clear to one of ordinary skill in the art that there are many equivalent configurations and devices that may be advantageously used in the present invention.

The device can also optionally plug into a wall for battery recharging. To recharge the batteries, for example, a lithium battery charger may be used, such as, but not limited to, the LT1510 from Linear Technologies. Running directly of the wall or powering the battery charger requires an AC to DC power supply such as, but not limited to, the JAMECO DFU240050F1121 (24V, 500 mA) for test and research or the GLOBTEK TR9CG1200LCP-Y-MED (15V, 1.2 A) for medical use. While specific devices and configurations are used in this example implementation, it will again be clear to one of ordinary skill in the art that there are many equivalent configurations and devices that may be advantageously used in the present invention.

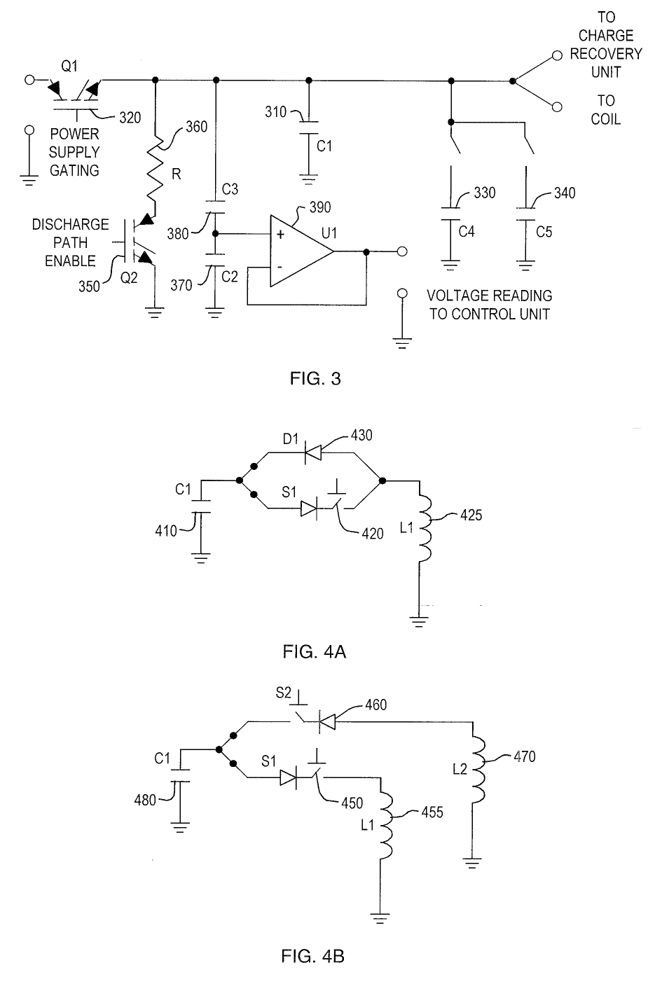

FIG. 3 is a circuit diagram of an exemplary embodiment of an energy storage unit, according to one aspect of the present invention. The energy storage unit of FIG. 3 comprises capacitor 310 (C1) that stores the energy coming from the power supply unit until it is needed to generate a stimulation pulse and additional circuitry to discharge it and measure its voltage. Since the voltage/current coming from the power supply is not continuous, the charge on the capacitor should not be allowed to escape back to the power supply. This is achieved by using a circuit element that allows conduction in one direction only, such as, but not limited to, a semiconductor diode (shown in FIG. 2 for a standard flyback transformer) or by isolated gate bipolar transistor (IGBT) 320 in FIG. 3 (Q1), a type of transistor that when properly used permits conduction in one direction only and can control the flow of current in the allowed direction. Since high current/voltage-rating IGBTs can be bulky and expensive, it is possible to use many low-rating IGBTs in parallel at a lower-than-maximum voltage in order to simulate a high current/voltage-rating IGBT. Some IGBTs come with a freewheeling diode to allow backward conduction. Those require an additional diode in series to prevent the built in diode from conducting. In the prototype implementation, an IRG4PSH71U from International Rectifier was used, which does not require the additional diode.

Capacitor 310 (C1) may comprise a single capacitor or, alternatively, a combination of capacitors connected in series and/or parallel in order to reach the desired voltage rating and total capacitance. The energy storage unit may further comprise many independent units of capacitors (e.g., C4 330, C5 340) connected together in parallel by using, for example, but not limited to, switches, relays, or solid state devices. All the capacitors, like the primary one, can be implemented as a series and/or parallel combination of physical components. The prototype implementation employs high current switches. Since parallel capacitances add, by switching in and out units the total capacitance of the energy storage unit can be changed, thereby changing the length of the pulse. In the prototype implementation, series parallel combinations of 25 uF, 450V low leakage film capacitors, p/n B32676 from Epcos are employed. Discharge of the capacitors is needed when the device is turned off, or in certain cases when units are switched in and out, and can be achieved by, for example, but not limited to, a resistive discharge path, usually disconnected but at times enabled by the control unit. In the prototype implementation, IGBT 350 (Q2) is used in series with resistor 360 (R) to control discharge.

Measuring the voltage on the energy storage unit can be accomplished by conventional methods, such as, but not limited to, using an analog to digital converter or measuring the charging time of an RC circuit to a fixed voltage, which is useful and easy to do with a microcontroller, as in the prototype. To get the high voltage into the range to be usable with such methods, a capacitive voltage divider (C2 370 and C3 380) is used, with output is buffered by a FET operational amplifier or buffer 390 (U1). When the capacitor is charged to the desired voltage, the power supply is disabled. In the prototype, the oscillator feeding the flyback transformer shuts off and the gating IGBT is put in non-conducting mode, thereby disabling the charging portion of the circuit. When it is desirable for the stimulator to be discharged, either because it needs to be totally discharged or because a lower power setting is chosen and too much voltage is already on the capacitor, the power supply is disabled, and the discharge path is enabled for long enough to leave the desired voltage in the energy storage unit, via a simple feedback controller. Such controller may be implemented electronically or in software within the control unit, as in the prototype.

The energy storage unit is preferably connected to the magnetic stimulation coil and the charge recovery system by a series of electrical circuit elements that permit conversion of the energy stored in the capacitor to energy stored in a strong current (>1 kA) in the stimulation coil, which from a circuit standpoint is an inductor, back to energy stored as a voltage in the capacitor of the energy storage unit. There are multiple ways to perform this procedure known in the art of the invention, with the preferred manner being selected according to the type of stimulus that is desired.

The modular design of the present invention facilitates changing only the charge recovery unit in order to make the device produce any kind of stimulation, keeping all of the other components the same. The behavior of the control unit needs to be reconfigured to do this. In the prototype implementation, which uses a PIC microcontroller, separate software is used to perform the appropriate sensing and control tasks. The connection disconnection of circuit elements during operation of the device is achieved using solid state switches such as, but not limited to, power MOSFETs, IGBTs, thyristors, and GTOs. The prototype implementation, employs IGBTs (the same model as for the energy storage unit) in parallel in order to carry enough current. This permits the device to be much smaller than if a higher-rated but bulkier and harder to operate IGBTs were used.

The circuits in the prototype use a unidirectional switch, which is represented by a voltage-controlled switch, followed by an ideal diode. Examples of unidirectional switches are, but are not limited to, Gate Turn Off Thyristors (GTO) or IGBTs. Power MOSFETs are unidirectional, but because of their structure always conduct reverse current, so for use in the circuits of the present invention they typically need some element to block reverse current, such as a diode in series. In the case where the switch needs to be turned off only when the current through it goes to zero, which happens for some switches in some designs, more devices are available for use, such as, but not limited to, Silicon Controlled Rectifiers (SCRs)/Thyristors or TRIACS.

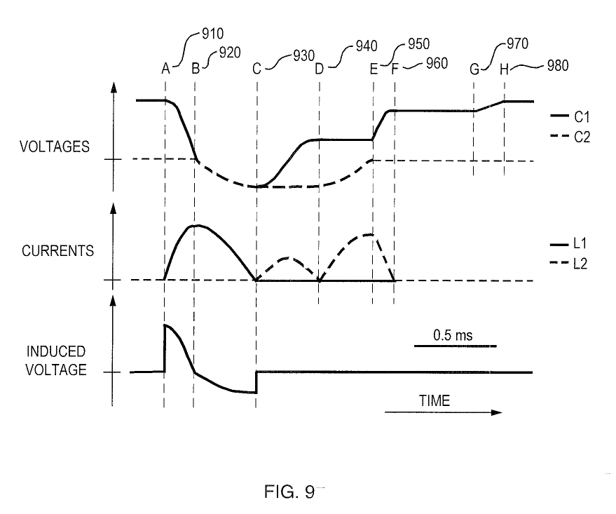

The charge recovery unit is configured to produce the desired stimulation type or pattern. One of the simplest simulation types is biphasic, where the induced current into the brain swings symmetrically in both directions. Multiphasic stimulation is a succession of biphasic stimulations. Also interesting and useful is monophasic stimulation, where the pulsed induced currents are designed to stimulate predominantly in one direction. Several implementations of these stimulation methods are possible. The prototype embodiment has been employed for the following types of stimulation: multiphasic, biphasic (with second inductor to restore polarity), monophasic, and monophasic with shared capacitors. FIGS. 4A-B-FIG. 9 present circuit and timing diagrams for circuits that produce these stimulation types. In these exemplary circuits, the energy storage unit is represented as a capacitor, but it is understood that the rest of the energy storage unit apparatus is present in the real device, and the omission of the remaining circuitry from the circuit diagrams is only for the purpose of clarity. Also, when a second capacitor/capacitive unit is present in the device, it is to be understood that the voltage of this unit can be measured by a circuit similar to the one for the energy storage unit, even though, for clarity, the measurement circuit is not shown.

FIGS. 4A and 4B are circuit diagrams of exemplary embodiments of a multiphasic stimulator and a biphasic stimulator, respectively, according to one aspect of the present invention. In FIG. 4A, which is adapted for multiphasic stimulation, energy storage unit 410 (C1) is charged to the appropriate level for the desired pulse strength via the power supply unit. Then the control unit disconnects the power supply unit and closes switch 420 (S1). At this point, energy storage unit 410 and coil 425 (L1) are connected in a LC circuit. The energy in energy storage unit 410 will be converted into current in the coil, and back into negative voltage on energy storage unit 410. At that point, D1 430 starts conducting and the process repeats, so that the voltage on energy storage unit 410 is positive again. Now the power supply unit is briefly enabled to replenish the energy loss in the process and bring back energy storage unit 410 to full charge, and the cycle is repeated. Multiphasic stimulation is of limited research or therapeutic use, but it is the simplest kind of stimulation, and can be easily implemented using the present invention. Switch 420 S1 can be implemented with an SCR or similar device as well, and the combination S1 D1 can be constituted by a unique device that conducts backwards, such as, but not limited to, a power MOSFET or an IGBT with a freewheeling diode.

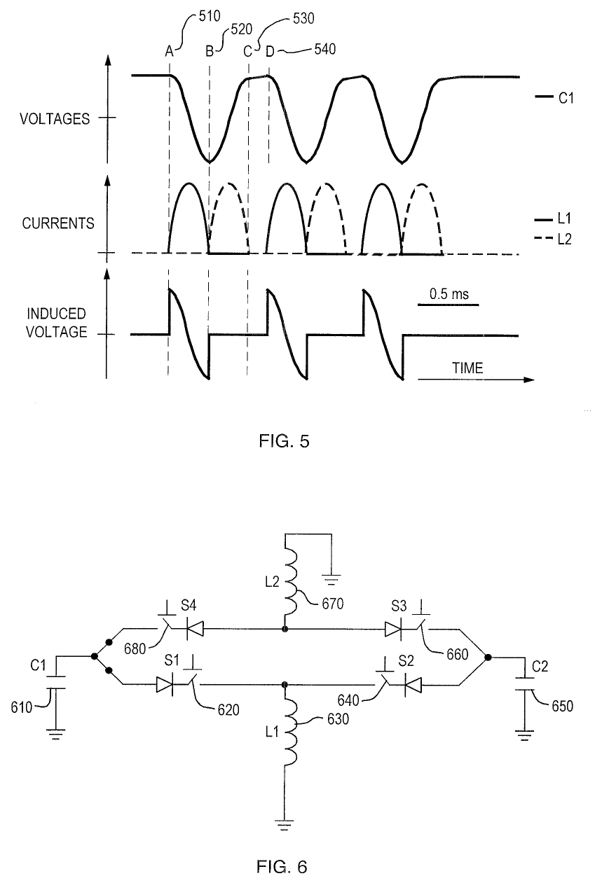

As shown in FIG. 4B, a biphasic stimulator can be implemented with two switches 450, 460 (S1, S2) and an additional high current inductor 470 (L2). The typical timing diagram for a biphasic stimulator is shown in FIG. 5. Referring to FIGS. 4B and 5, the device starts at the desired voltage on the energy storage unit 480 (C1), and it is triggered at time A 510 by the control unit, which makes sure that the power supply unit is disconnected. S1 450 is closed and current starts to flow in stimulation coil 455 (L1), and it keeps flowing until time B 520, when the voltage on C1 480 is at its minimum. At this point, S1 450 is opened and S2 460 is closed (optionally, some time can be waited before closing S2 460 if necessary), so that current starts flowing in L2 470, and it flows until time C 530, when a positive voltage is again on C1 480. At this point the power supply unit can be used to replenish the energy lost (again, this can optionally wait) and at time D 540, once the voltage is back to the target level, another stimulation pulse can be discharged.

To achieve monophasic stimulation, different durations are needed for the raising and falling part of the pulse. This is accomplished by means of a multi-step cycle. Energy is stored in the initial capacitor, the stimulating coil is connected, and the capacitor discharges into it. When the current in the coil is maximum, the capacitor is disconnected, and a different energy storage unit is connected, with a different capacitance and therefore different time constant. Components of the first unit can also be shared by the second one; they need not necessarily to be separate. The stimulating coil discharges into the second energy storage unit. Once all the energy is in the second unit, it is disconnected from the coil, and connected to another inductor (not the same coil, unless another stimulation pulse is desired). The second unit discharges into the inductor. When the current in the inductor is maximum, it is disconnected from the second unit and connected to the first one. The inductor discharges into the energy storage unit. Once the current in the inductor is zero, it is disconnected, and most of the energy is recovered in the energy storage unit.

Monophasic stimulation, stimulation predominantly in one direction, is achieved in existing devices by damping the pulse with a dissipative element, so that there is no or little energy left in the device (except in the form of heat of the dissipative element) after one pulse and that is the main reason for bulkiness and high power consumption of the other devices. The monophasic pulses of this implementation take advantage of the fact that nerve cells require a threshold electric field to fire, therefore asymmetric pulses may be constructed that do not require energy dissipation by having a short, high intensity induced field in one direction, and a longer but less intense field in the opposite direction. If the longer part of the pulse has an intensity below the threshold, it will not stimulate significantly the underlying neurons or nerves.

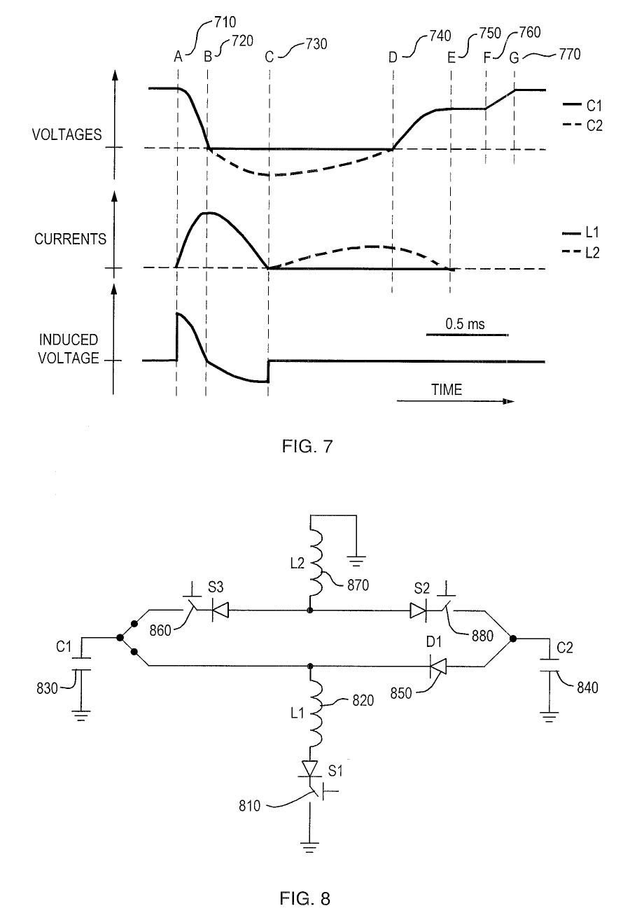

One possible circuit to implement these kinds of pulses is shown in FIG. 6, which is a circuit diagram of an exemplary embodiment of a monophasic stimulator, according to one aspect of the present invention. FIG. 7 is a typical timing diagram for the stimulator of FIG. 6. Referring to FIGS. 6 and 7, initially, energy storage unit 610 (C1) is kept at the desired voltage. When the pulse is needed, switch 620 (S1) is closed (at time A 710) while the power supply unit is disconnected. C1 610 will start discharging into coil 630 (L1). Once the voltage on C1 610 gets to zero, switch 640 (S2) is closed and S1 620 is opened with the least delay between them (time B 720). Now C2 650, initially with no voltage, is connected to L1 630, which transfers the energy stored as current into energy on C2 650. Once all the energy is transferred (at time C 730), S2 640 can be opened. No precise timing is required if the switch is unidirectional, as in this example. If C2 650 has a different capacitance than C1 610, the time constant that it will make with stimulating coil L1 630 will be different, and therefore the pulse shape will be asymmetric. To achieve monophasic stimulation during the raising edge of the current through coil 630, C2 650 must be larger than C1 610. Conversely, C1 610 being larger than C2 650 allows for stimulation during the falling edge. In the first case, for example, C2 650 must be large enough that the induced field when L1 630 is charging C2 650 is below the threshold, otherwise biphasic stimulation is achieved. Next (possibly after waiting, if needed), switch 660 (S3) is closed, and C2 650 will discharge through L2 670, the auxiliary inductor. Once the voltage of C2 650 reaches zero (at time D 740), switch 680 (S4) is closed and S3 660 is opened, as fast as possible. Now the energy stored in L2 670 will be completely transferred back to C1 610 at time E 750, and S4 680 can be opened. Because of losses, the voltage on C1 610 now is less than the initial voltage, so the power supply unit is enabled (at time F 760) until the target voltage is reached (at time G 770), and the device is ready for the next pulse. In this embodiment, switches S3 660 and S4 680 can be advantageously implemented by SCRs or similar devices.

FIG. 8 is a circuit diagram of an exemplary embodiment of a monophasic stimulator design using the energy storage unit as part of the secondary capacitor, according to one aspect of the present invention. FIG. 9 depicts a typical timing diagram for the stimulator of FIG. 8. This design achieves the same results as the previous one, but it uses part of the capacitance of C1 to store the energy after the second phase of the cycle. Referring to FIGS. 8 and 9, once switch 810 (S1) is closed (at time A 910), coil 820 (L1) and energy storage unit 830 (C1) form an LC circuit. When the voltage of C1 830 goes below ground, C2 840 (initially with no voltage) is effectively connected in parallel to C1 830 by the action of diode 850 (D1), and the LC circuit now has a different time constant and reaches the maximum negative voltage at time C 930. At this point, S1 810 can be opened. S1 810 can be implemented by an SCR or similar device. The resulting pulse is similar to the one obtained with the previous method, but C2 840 needs to be smaller to achieve the same result. Because of diode 850, the voltage of C2 840 needs always to be smaller than the voltage of C1 830, therefore switch 860 (S3) is closed (at time C 930, possibly with waiting) and L2 870 reverses the voltage on C1 830, which now is positive (at time D 940). Keeping S3 860 closed, switch 880 (S2) is now closed too, and the voltage on C2 840 is converted into current on L2 870. Once the voltage of C2 840 reaches zero (at time E 950), S2 880 is swiftly opened, forcing the current in L2 870 through S3 860 onto C1 830. At time F 960, the transfer is complete, and the power supply unit can be enabled again (at time G 970) until the desired starting voltage is replenished (time H 980), and the device is ready for another pulse.

In a preferred embodiment, the coil unit comprises a conductive coil that produces a strong magnetic field when the pulse of current generated by the device traverses it. The field ideally should be mostly external to the coil itself, so that when the coil is brought in contact with the user's head, most of the field ends up within the skull. The best designs appear to have a FIG. 8 or similar shape, such as rectangular (where the two loops are rectangular instead of circular). To increase the magnetic flux density towards the user's head, the coil may be augmented with a ferromagnetic core, such as, for example, silicon steel. Two model coil designs implemented as prototypes to test the present invention were made of 10-gauge copper magnet wire, insulated with heavy amidester coating, and had two 2.5-15 cm diameter coils arranged in a figure-8. Many other coil geometries exist, and any coils with the same inductance can be interchanged with one another without alteration of any other part of the circuit, making this also a modular aspect of the circuit. The first prototype implementation, a 2.5″ model, specifically employs a coil wound around a core made of 3% silicon steel, tape-wound into a ring, in order to vastly increase the inductance of the coil while lowering the peak voltages and currents required to be reached by the rest of the circuit. Three such tape-wound cores (Custom Transformer Core, ID 0.5″, OD 3″ height 0.5″, Alpha Core) are epoxyed together to adequate thickness with high-temperature conductance, insulating epoxy (MG Thermally Conductive Epoxy 832TC). Cores are then cut into half-toroidal shape using the waterjet cutter. The second implementation is a ‘CD’ shaped coil (similar to a figure-8 shape, but pinched in the middle), made of solid 11 gauge copper wire, with no magnetic core. The diameter of the loops is 2-10 cm. A smaller coil has less flux, and thus requires a higher current to drive; a larger coil requires less current.

Optional features of the present invention include, but are not limited to, the capacity to recharge the battery via remote power, such as, but not limited to, through an RF inductive coil, including through the same coil used to do the stimulation, making the coil into an array of coils, each individually actuatable, for more focal stimulation, and replacing the coils with a mesh electrode of copper wires that can go under the hair, for invisible and fashionable wearable brain stimulation. The TMS machines themselves can also be considered as individual modules. This modularity allows TMS machines to be assembled together to make bigger TMS machines, with multiple independent coils, and hence with more flexibility.

The customizable and programmable control unit allows multiple TMS machines to be synchronized precisely and to be controlled as a whole by an external device, such as, but not limited to, by a personal computer or another microcontroller. This can lead to novel stimulation patterns made by stimulating different parts of the brain with a precise timing. For example, a frontal attentional area and an early sensory area can be activated, in order to strengthen association between attentional areas and sensory areas. Many brain disorders are associated with decreased connectivity or strength between different regions; here we enable the ability to strengthen these links by stimulating multiple ones in close conjunction (e.g., stimulating region A 10 ms before region B to strengthen the A→B projection, or stimulating region A 10 ms behind region B to weaken the A→B projection). The present invention enables the ability to stimulate one or more regions of the brain at one or more times, relative to one another, because of the ability to assemble TMS individual modules according to the present invention into emergent structures. The device may also be used in a patient-customized way, in order to adapt to the brain structures of the patient, by positioning the set of coils over sets of brain regions. It is thus envisioned that the present invention has many uses, including, but not limited to diagnosis or treatment of epilepsy, Parkinson's, migraine, depression, schizophrenia, tinnitus, or any other neurological or psychiatric disorder, as well as use of such a device to augment memory, intelligence, or other human attributes.

While a preferred embodiment is disclosed, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

What is claimed is:

1. A modular transcranial magnetic stimulation device, comprising:
power supply unit, the power supply unit being able to generate specified voltages from a low voltage supply and being externally controllable;
energy storage unit, the energy storage unit being able to store a voltage received from the power supply unit until it is needed to generate a stimulation pulse;
magnetic stimulation coil unit, the magnetic stimulation coil unit storing energy received from the energy storage unit as a strong current available for application of magnetic stimulation to a patient;
charge recovery unit, the charge recovery unit being able to convert energy stored as current in the magnetic stimulation coil unit back to energy stored as a voltage in the energy storage unit; and
control unit, for controlling the operations of the power supply unit, the energy storage unit, and the charge recovery unit.

2. The device of claim 1, the energy storage unit further comprising discharge circuitry for discharging the stored voltage at the direction of the control unit.

3. The device of claim 1, the power supply unit comprising a flyback transformer.

4. The device of claim 1, wherein the device is portable.

5. The device of claim 1, wherein the device is reconfigurable.

6. The device of claim 1, the energy storage unit comprising a plurality of capacitors.

7. The device of claim 1, the control unit comprising a microcontroller.

8. The device of claim 1 the charge recovery unit being configured such that the magnetic stimulation delivered to the patient is biphasic.

9. The device of claim 1 the charge recovery unit being configured such that the magnetic stimulation delivered to the patient is multiphasic.

10. The device of claim 1, the charge recovery unit being configured such that magnetic stimulation delivered to the patient is monophasic.

11. The device of claim 1, the magnetic stimulation coil unit comprising a plurality of coils.

12. A modular transcranial magnetic stimulation device, comprising a plurality of devices according to claim 1.
